(19) United States
(12) Patent Application Publication
(10) Pub. No.: US 2019/0325312 A1
(43) Pub. Date: Oct. 24, 2019

(54) COMPUTER-READABLE RECORDING MEDIUM, MACHINE LEARNING METHOD, AND MACHINE LEARNING APPARATUS

(71) Applicant: FUJITSU LIMITED, Kawasaki-shi (JP)

(72) Inventors: Tatsuru MATSUO, Kawasaki (JP); Izumi NITTA, Kawasaki (JP)

(73) Assignee: FUJITSU LIMITED, Kawasaki-shi (JP)

(21) Appl. No.: 16/380,537

(22) Filed: Apr. 10, 2019

(30) Foreign Application Priority Data
Apr. 20, 2018 (JP) 2018-081907

(51) Int. Cl. G06N 3/08; G06N 20/00; G06Q 10/10; G16H 50/30

(52) U.S. Cl. CPC G06N 3/084; G16H 50/30; G06Q 10/1091; G06N 20/00

(57) ABSTRACT

A learning apparatus receives time-series data including a plurality of items and including a plurality of records corresponding to a calendar. The learning apparatus generates tensor data, based on the time-series data, including a tensor which is set calendar information and each of the plurality of items as mutually-different dimensions. With respect to a learning model that performs a tensor decomposition on input tensor data and that inputs a result of the tensor decomposition to a neural network, the learning apparatus performs a deep learning process on the neural network and learning a method of the tensor decomposition by using the tensor data as the input tensor data.

### CROSS-REFERENCE TO RELATED APPLICATION

This application is based upon and claims the benefit of priority of the prior Japanese Patent Application No. 2018-081907, filed on Apr. 20, 2018, the entire contents of which are incorporated herein by reference.

### FIELD

The embodiments discussed herein are related to a computer-readable recording medium, a machine learning method, and a machine learning apparatus.

### BACKGROUND

To avoid situations where workers take an administrative leave of absence (have a medical treatment), a prediction is made for workers who may be in poor mental health a number of months later on the basis of worker attendance record data so that appropriate steps can be taken (e.g., offer counseling) at early stages. According to a commonly-used method, dedicated staff visually goes through data to look for workers corresponding to a certain working state having any of characteristic patterns that are characteristic to the occurrence of poor mental health, such as frequent business trips, long overtime hours, sudden consecutive absences, absences without notice, and a combination of any of these. It is difficult to clearly define these characteristic patterns, partly because different dedicated staff members use different criteria. In recent years, an endeavor has been made to mechanically make a prediction for the assessments to be made by the dedicated staff, by learning the characteristic patterns characterizing poor mental health, through a machine learning process that uses a decision tree, a random forest, a Support Vector Machine (SVM), or the like.

Patent Literature 1: Japanese Laid-open Patent Publication No. 2016-151979

### SUMMARY

According to an aspect of an embodiment, a non-transitory computer-readable recording medium stores therein a machine learning program that causes a computer to execute a process. The process includes receiving time-series data including a plurality of items and including a plurality of records corresponding to a calendar; generating tensor data, based on the time-series data, including a tensor which is set calendar information and each of the plurality of items as mutually-different dimensions; and with respect to a learning model that performs a tensor decomposition on input tensor data and that inputs a result of the tensor decomposition to a neural network, performing a deep learning process on the neural network and learning a method of the tensor decomposition by using the tensor data as the input tensor data.

The object and advantages of the invention will be realized and attained by means of the elements and combinations particularly pointed out in the claims.

It is to be understood that both the foregoing general description and the following detailed description are exemplary and explanatory and are not restrictive of the invention.

### BRIEF DESCRIPTION OF DRAWINGS

FIG. 1 is a drawing for explaining an example of an entire machine learning process according to a first embodiment;

FIG. 2 is a drawing illustrating an example of a relationship between a graph structure and a tensor;

FIG. 3 is a drawing illustrating an example of a process of extracting a partial graph structure;

FIG. 4 is a drawing for explaining an example of a learning process using a deep tensor;

FIG. 5 is a functional block diagram illustrating a functional configuration of a learning apparatus according to the first embodiment;

FIG. 6 is a drawing illustrating an example of worker attendance record data stored in a worker attendance record data database (DB);

FIGS. 7A and 7B are drawings illustrating examples of a label setting process;

FIG. 8 is a drawing for explaining a tensor data generating process;

FIG. 9 is a drawing for explaining a specific example of a tensor creating process;

FIG. 10 is a flowchart illustrating a flow in a learning process;

FIG. 11 is a drawing for explaining advantageous effects;

FIG. 12 is a drawing for explaining an example of a hardware configuration;

FIG. 13 is a drawing for explaining a data format used in a commonly-used machine learning process;

FIG. 14 is a drawing for explaining a learning method used in the commonly-used machine learning process; and

FIG. 15 is a drawing for explaining problems of the commonly-used machine learning process.

### DESCRIPTION OF EMBODIMENTS

However, in commonly-used machine learning processes, when data is input to a machine learning model, the data is converted into a form compliant with the input format of the machine learning model. Accordingly, relationships that have not been recognized at the time of the data conversion are lost during the data conversion. As a result, the learning process is not properly performed.

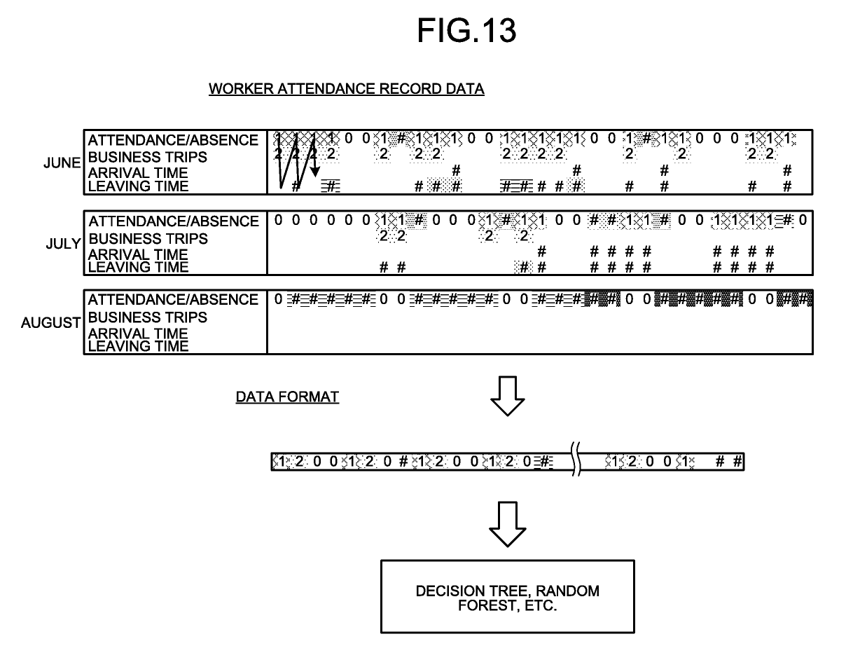

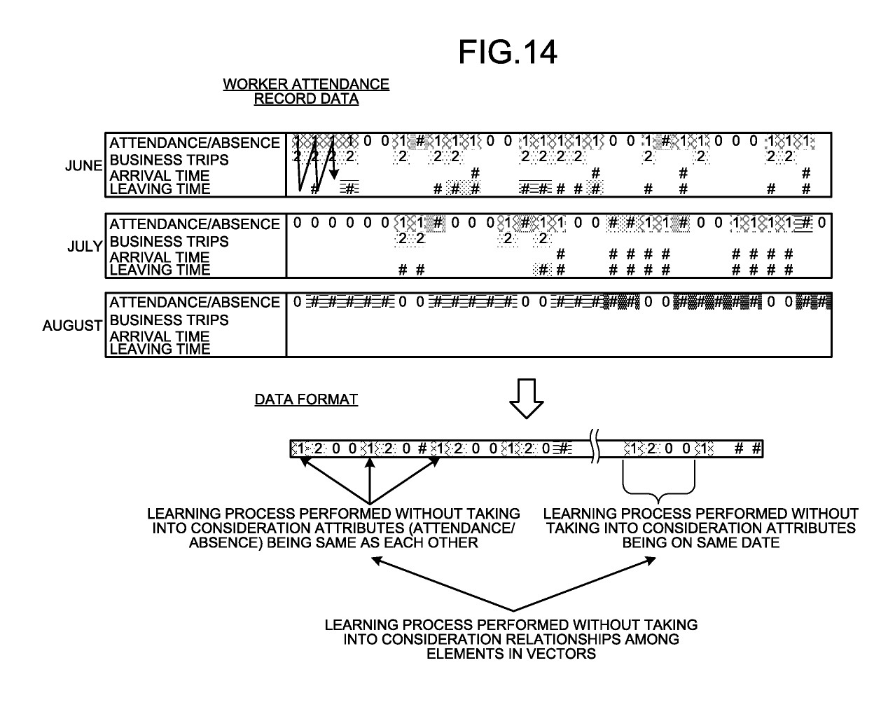

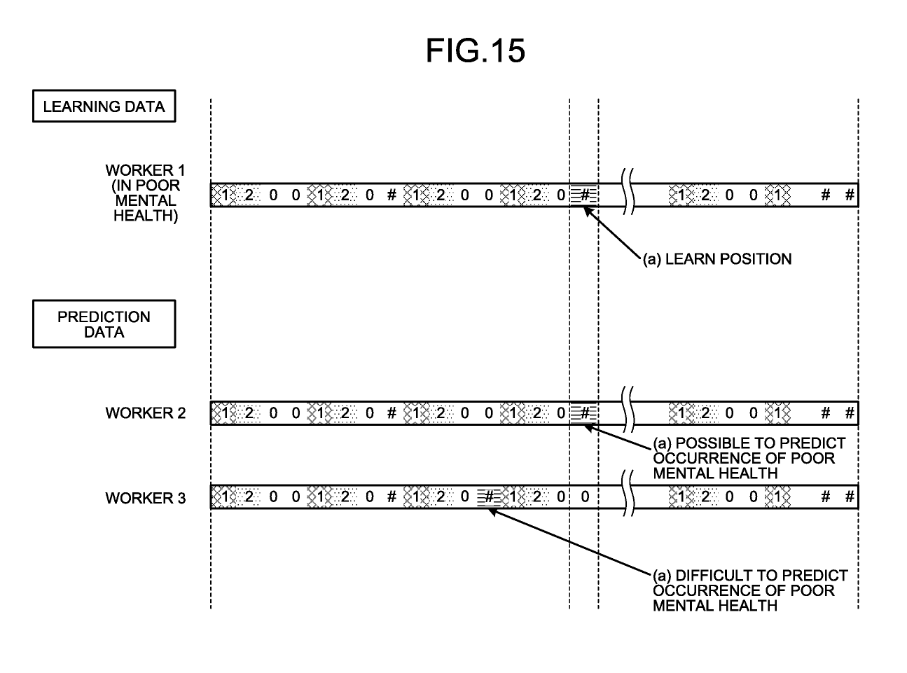

More specifically, in commonly-used machine learning processes, feature vectors are created from worker attendance record data. However, a learning process and a predicting process are unfortunately performed while missing characteristics of a calendar which the worker attendance record data has, such as attributes and relevance of elements of the worker attendance record data. Next, problems of a commonly-used machine learning process will be explained, with reference to FIGS. 13 to 15. FIG. 13 is a drawing for explaining a data format used in the commonly-used machine learning process. FIG. 14 is a drawing for explaining a learning method used in the commonly-used machine learning process. FIG. 15 is a drawing for explaining problems of the commonly-used machine learning process. The numerals used in the worker attendance record data illustrated in FIGS. 13 to 15 are pieces of information indicating categories such as attended=0; an administrative leave of absence=1; a saved-up vacation=2; a paid vacation=3.

As illustrated in FIG. 13, the commonly-used machine learning process is based on the premise that fixed-length feature vectors are input. Training data is generated by expressing, by using vectors, the status on each day in the worker attendance record data, in the order indicated by the arrow in the worker attendance record data. More specifically, the values set to the elements are sequentially expressed by using the vectors, for example, in the order of: the attendance/absence status on June 1st, the business trip status on June 1st, the arrival time information (the time at which the worker arrived at work) on June 1st, the leaving time information (the time at which the worker left work) on June 1st, the attendance/absence status on June 2nd, the business trip status on June 2nd, the arrival time information on June 2nd, and the leaving time information on June 2nd.

As explained above, the data format of the training data is simply vector information and does not have attribute information of the elements of the vectors. It is therefore not possible to distinguish which values correspond to attendance/absence information and which values correspond to business trip information. For this reason, as illustrated in FIG. 14, the learning process is performed without taking into consideration certain attributes (attendance/absence) being the same as each other and without taking into consideration certain attributes being on mutually the same date. As a result, the learning process is performed without taking into consideration the relationships among the elements in the vectors.

For example, as illustrated in FIG. 15, let us discuss an example in which a cause of a worker being in poor mental health can be analyzed as a situation where worker attendance record data includes an attribute value (a) exhibited in training data corresponding to the worker attendance record data of a worker 1 who is in poor mental health. In this situation, a learning apparatus performs a learning process by using the position of the attribute value (a) within the vector as a characteristic pattern. Accordingly, from prediction target data corresponding to the worker attendance record data of another worker 2, the learning apparatus detects the attribute value (a) in the same position as the position in the worker attendance record data of the worker 1 that has been learned and is thus able to predict the occurrence of poor mental health. However, although prediction target data corresponding to the worker attendance record data of yet another worker 3 contains the attribute value (a) having the same value as the attribute value (a) in the worker attendance record data of the worker 1 that has been learned, because the position in which the attribute value (a) is included is different, it is difficult for the learning apparatus to predict the occurrence of poor mental health.

Preferred embodiments will be explained with reference to accompanying drawings. The present disclosure is not limited to the exemplary embodiments. Further, it is possible to combine any of the embodiments together as appropriate, as long as no conflict occurs.

### [a] First Embodiment

### An Overall Example

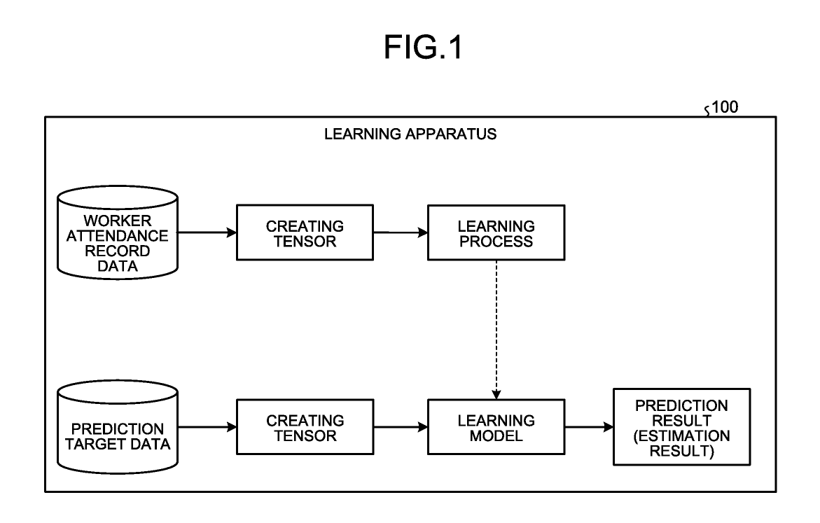

FIG. 1 is a drawing for explaining an example of an entire machine learning process according to a first embodiment. As illustrated in FIG. 1, a learning apparatus 100 according to a first embodiment is an example of a machine learning apparatus and is an example of a computer apparatus that generates a learning model by performing a machine learning process on worker attendance record data including various daily statuses of each worker such as arrival and leaving times, vacations taken, business trips, and the like, and predicts whether a certain worker subject to the predicting process will need a medical treatment (an administrative leave of absence) or will not need a medical treatment (an administrative leave of absence) on the basis of the worker attendance record data of the worker, by using a learning model resulting from the learning process. In the following sections, an example will be explained in which the learning apparatus 100 performs the learning process and the predicting process; however, it is also acceptable to have the learning and the predicting processes performed by separate apparatuses.

More specifically, the learning apparatus 100 generates the learning model by using a deep tensor that implements a Deep Learning (DL) process on data having a graph structure while using, as supervised data, worker attendance record data of one or more workers who were in poor health and took an administrative leave of absence (labeled as “a medical treatment: Yes”) and worker attendance record data of one or more workers who are in normal health and has not taken an administrative leave of absence (labeled as “a medical treatment: No”). After that, by using the learning model to which results of the learning process are applied, estimation of accurate events (labels) is realized with data having a new graph structure.

For example, the learning apparatus 100 receives worker attendance record data including a plurality of items and including a plurality of records corresponding to a calendar. From the worker attendance record input data, the learning apparatus 100 generates tensor data by creating a tensor while using calendar information and each of the plurality of items as mutually-different dimensions. Further, with respect to a learning model that performs a tensor decomposition by using the tensor data as an input and that further inputs a result of the classification process to a neural network, the learning apparatus 100 performs a deep learning process on the neural network and learns a method of the tensor decomposition. In this manner, the learning apparatus 100 generates the learning model that classifies data into categories of “a medical treatment: Yes” and “a medical treatment: No” from the tensor data of the worker attendance record data.

After that, the learning apparatus 100 generates tensor data by similarly creating a tensor from the worker attendance record data of a worker subject to the assessment and inputs the generated tensor data to the learning model resulting from the learning process. Further, the learning apparatus 100 outputs an output value indicating a prediction result as to whether the worker will be classified as “a medical treatment: Yes” or “a medical treatment: No”.

Next, the deep tensor will be explained. The deep tensor is a deep learning scheme that uses a tensor (graph information) as an input. The deep tensor is designed to learn a neural network and to automatically extract a partial graph structure that will contribute to the assessment. The extracting process is realized by learning the neural network and learning parameters of the tensor decomposition performed on input tensor data.

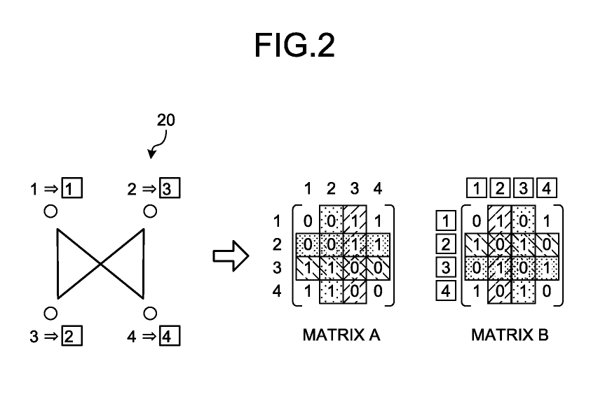

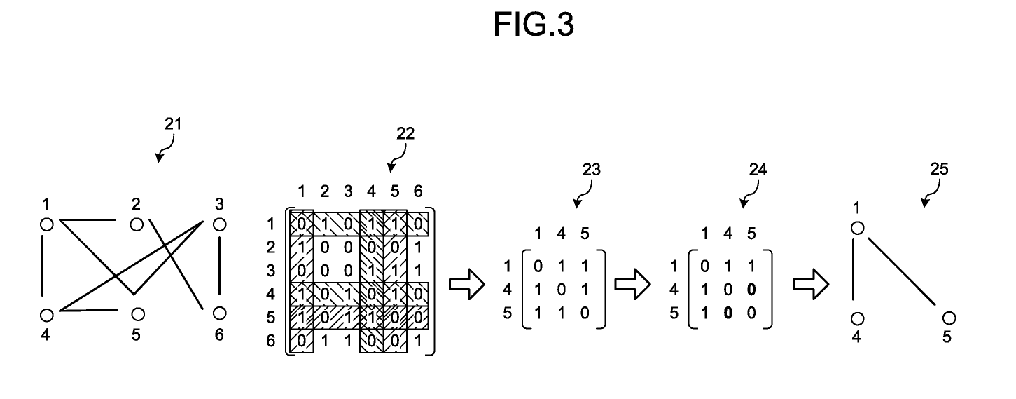

Next, the graph structure will be explained with reference to FIG. 2 and FIG. 3. FIG. 2 is a drawing illustrating an example of a relationship between the graph structure and a tensor. In a graph 20 illustrated in FIG. 2, four nodes are connected by edges each indicating a relationship between nodes (e.g., “a correlation coefficient is equal to or larger than a predetermined value”). In this situation, it is indicated that any nodes that are not connected to each other by an edge have no relationship. When the graph 20 is expressed by using second-order tensors, i.e., matrices, for example, the matrix expression based on the number on the left-hand side of a node is expressed by a “matrix A”, whereas the matrix expression based on the number on the right-hand side of a node (the number in a square) is expressed by a “matrix B”. These components of the matrices are expressed with “1” when the nodes are joined (connected) to each other and are expressed with “0” when the nodes are not joined (connected) to each other. In the following sections, this type of matrices may be referred to as adjacent matrices. In this situation, it is possible to generate the “matrix B” by simultaneously permuting the second and third rows and the second and third columns of the “matrix A”. In the deep tensor scheme, processing is performed while ignoring the differences in the sequential order by using such permutation process. In other words, in the deep tensor scheme, the “matrix A” and the “matrix B” are treated as mutually the same graph, while the sequential order thereof is ignored. It is possible to apply the same process to tensors of the third or higher order.

FIG. 3 is a drawing illustrating an example of the process of extracting the partial graph structure. In a graph 21 illustrated in FIG. 3, six nodes are connected together with edges. It is possible to express the graph 21 as indicated in a matrix 22 by using the matrix (a tensor). It is possible to extract a partial graph structure by performing, on the matrix 22, a combination of: a calculation to switch specific rows and columns; a calculation to extract specific rows and columns; and a calculation to substitute non-zero elements in adjacent matrices with zero. For example, when the rows and the columns corresponding to “nodes 1, 4, and 5” in the matrix 22 are extracted, a matrix 23 is obtained. Subsequently, when the values between “nodes 4 and 5” in the matrix 23 are substituted with zeros, a matrix 24 is obtained. A partial graph structure corresponding to the matrix 24 is obtained as a graph 25.

The process of extracting the partial graph structure described above is realized with a mathematical calculation called a tensor decomposition. The tensor decomposition is a calculation to approximate an n-th order tensor that has been input by using a product of tensors of n-th or lower order. For example, the n-th order tensor that has been input is approximated by using a product of one n-th order tensor (called a core tensor) and tensors of which the quantity is equal to n and which are of lower order (which are usually second-order tensors (i.e., matrices) when n>2 is true). This classification process is not in one-to-one correspondence, and it is possible to arrange the core tensor to include an arbitrary partial graph structure of the graph structure expressed by the input data.

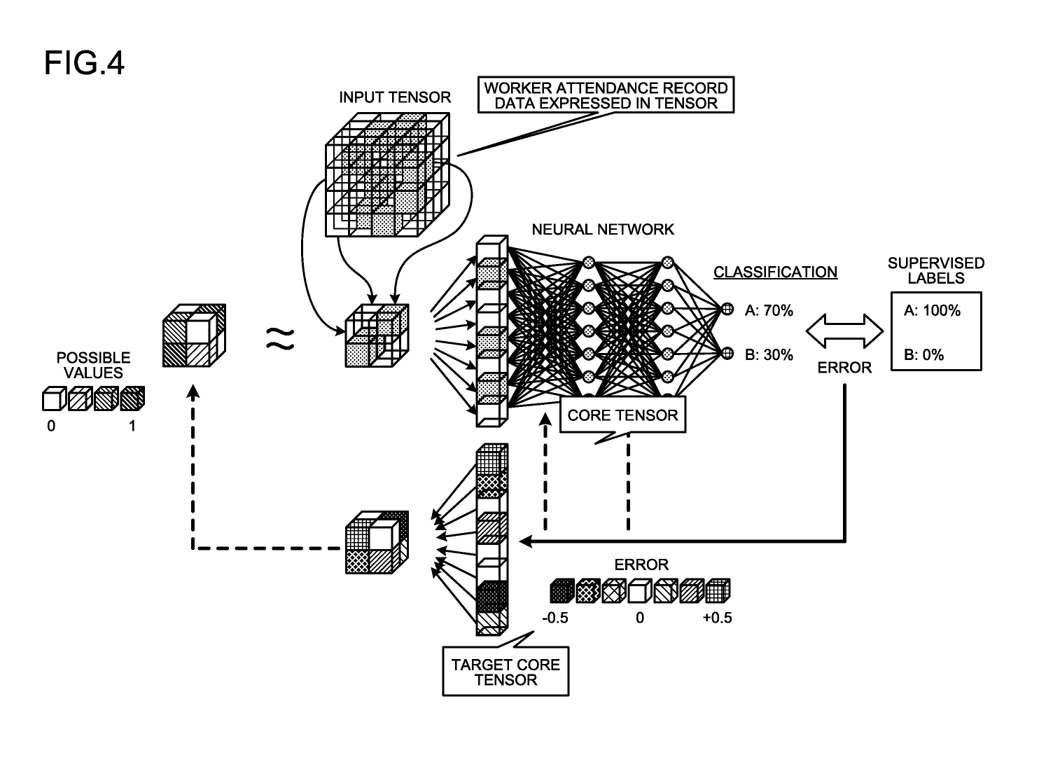

Next, a learning process using a deep tensor will be explained. FIG. 4 is a drawing for explaining an example of the learning process using a deep tensor. As illustrated in FIG. 4, the learning apparatus 100 generates tensor data from worker attendance record data to which a supervised label (a label A) indicating “a medical treatment: Yes” or the like is appended. Further, the learning apparatus 100 performs a tensor decomposition by using the generated tensor data as an input tensor and generates a core tensor that is similar to a target core tensor generated at random during the first session. After that, the learning apparatus 100 inputs the core tensor to a Neural Network (NN) and obtains a classification result (the label A: 70% and the label B: 30%). Subsequently, the learning apparatus 100 calculates a classification error between the classification result (the label A: 70% and the label B: 30%) and the supervised label (the label A: 100% and the label B: 0%).

In this situation, the learning apparatus 100 performs a learning process on a prediction model, by using an extended error propagation method obtained by extending an error backpropagation method. In other words, the learning apparatus 100 corrects various types of parameters of the NN so as to minimize the classification error by propagating the classification error to lower layers, with respect to an input layer, an intermediate layer, and an output layer of the NN. Further, the learning apparatus 100 propagates the classification error up to the target core tensor and further corrects the target core tensor so as to be approximated to a partial structure of the graph that will contribute to the predicting process, i.e., either a characteristic pattern indicating characteristics of workers who took an administrative leave of absence or a characteristic pattern indicating characteristics of workers who are in normal health. With this arrangement, in the optimized target core tensor, a partial pattern that will contribute to the predicting process has been extracted.

During the predicting process, it is possible to obtain a prediction result, by converting the input tensor into the core tensor (the partial pattern of the input tensor) by performing a tensor decomposition and further inputting the core tensor to the neural network. During the tensor decomposition, the core tensor is converted so as to be similar to the target core tensor. In other words, the core tensor having the partial pattern that will contribute to the predicting process is extracted.

A Functional Configuration

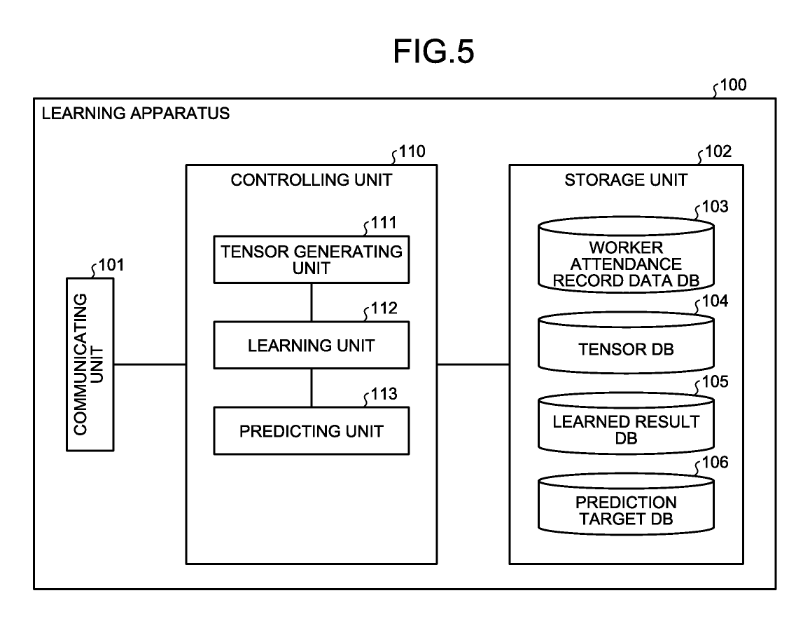

FIG. 5 is a functional block diagram illustrating a functional configuration of the learning apparatus 100 according to the first embodiment. As illustrated in FIG. 5, the learning apparatus 100 includes a communicating unit 101, a storage unit 102, and a controlling unit 110.

The communicating unit 101 is a processing unit that controls communication with other apparatuses and may be, for example, a communication interface. For instance, the communicating unit 101 receives an instruction to start a process, the worker attendance record data, and the like, from a terminal device of an administrator. Further, the communicating unit 101 outputs a result of a learning process, a result of a predicting process performed after the learning process, and the like, to the terminal device of the administrator.

The storage unit 102 is an example of a storage device storing therein computer programs (hereinafter “programs”) and data and may be configured by using a memory or a hard disk, for example. The storage unit 102 stores therein a worker attendance record data database (DB) 103, a tensor DB 104, a learned result DB 105, and a prediction target DB 106.

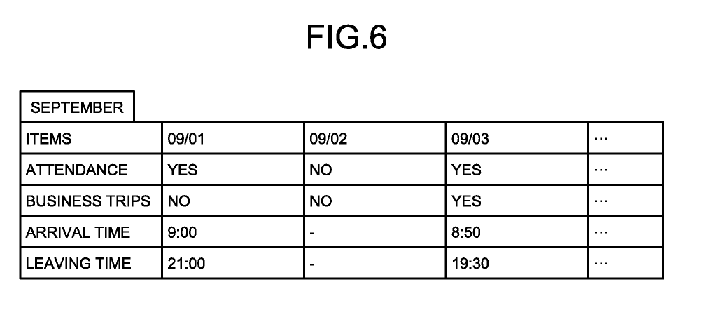

The worker attendance record data DB 103 is a database storing therein the worker attendance record data that is related to attendance of workers and the like and has been input thereto by a user or the like. The worker attendance record data DB 103 is an example of the time-series data. The worker attendance record data stored in the present example includes a plurality of items and includes a plurality of records corresponding to a calendar. Further, the worker attendance record data is obtained by expressing worker attendance records used in corporations in the form of data and may be obtained from any of various types of publicly-known worker attendance management systems or the like. FIG. 6 is a drawing illustrating an example of the worker attendance record data stored in the worker attendance record data DB 103. As illustrated in FIG. 6, the worker attendance record data is structured with records in units of days in each of the months (the months of the year) corresponding to the calendar. Each of the records stores therein, as worker attendance information, values of items such as “attendance/absence; a business trip: Yes/No; the arrival time; and the leaving time”, so as to be kept in correspondence with one another. The example in FIG. 6 indicates that “on September 1st, the worker did not have a business trip, arrived at work at 9:00, and left work at 21:00”.

For the item “attendance/absence”, for example, values indicating “attended”, “a medical treatment (an administrative leave of absence)”, “a saved-up vacation”, and “a paid vacation”, and the like may be set. For the item “a business trip: Yes/No”, values indicating whether or not the worker had a business trip may be set, so as to store therein one of the values corresponding to either “a business trip: Yes” or “a business trip: No”. In this situation, these values may be distinguished from one another by using numerical values. For example, it is possible to indicate the distinction as follows: “attended=0”; “a medical treatment=1” “a saved-up vacation=2”; and “a paid vacation=3”. Further, as for the units of the records in the worker attendance record data corresponding to the calendar, the records do not necessarily have to be in units of days and may be in units of weeks or months. Further, to accommodate situations where workers are allowed to take vacations in units of hours, it is also acceptable to set a value “an hourly vacation =4”. Further, the items serve as an example of the attributes.

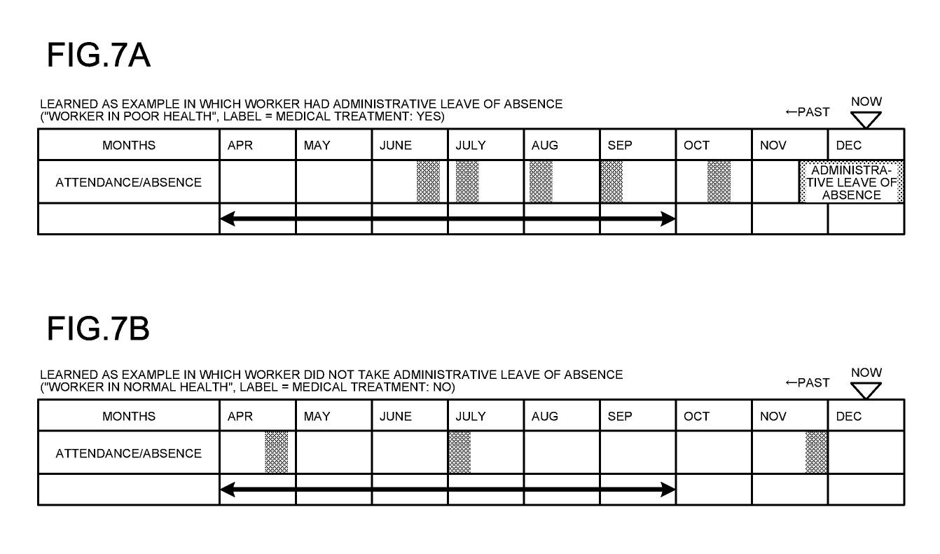

The worker attendance record data stored in the present example is learning data, and a supervised label is appended thereto. FIGS. 7A and 7B are drawings illustrating examples of a label setting process. FIG. 7A illustrates worker attendance record data of a worker who was in poor health and took an administrative leave of absence, and the label “a medical treatment: Yes” is appended thereto. FIG. 7B illustrates worker attendance record data of a worker who was in normal health and did not take an administrative leave of absence, and the label “a medical treatment: No” is appended thereto. For example, a tensor is created from worker attendance record data from six months as one piece of training data. It is possible to set the label “a medical treatment: Yes”, when a worker took an administrative leave of absence within the following three months and had an administrative-leave absence period. In contrast, it is possible to set the label “a medical treatment: No” when a worker did not take an administrative leave of absence within the following three months and did not have any administrative-leave absence period.

The tensor DB 104 is a database storing therein the tensors (the tensor data) generated from the worker attendance record data of the workers. The tensor DB 104 stores therein training data in which the tensors and the labels are kept in correspondence with each other. For example, as sets each made up of “tensor data and a label”, the tensor DB 104 stores therein “tensor data 1, the label ‘a medical treatment: Yes’”, “tensor data 2, the label ‘a medical treatment: No’”, and so on.

The items of the records in the learning data and the settings of the labels for the tensor data described above are merely examples. Besides the values and the labels indicating “a medical treatment: Yes” and “a medical treatment: No”, it is possible to use any of various values and labels capable of distinguishing whether workers in poor health are present or not, such as “a worker in poor health” and “a worker in normal health”; or “an administrative leave of absence: Yes” and “an administrative leave of absence: No”; or the like.

The learned result DB 105 is a database storing therein results of the learning process. For example, the learned result DB 105 stores therein assessment results (classification results) from the learning data obtained by the controlling unit 110, as well as the various types of parameters of the NN and the various types of parameters of the deep tensor that were learned through the machine learning process and the deep learning process.

The prediction target DB 106 is a database storing therein the worker attendance record data on which a prediction is to be made as to whether an administrative leave of absence will occur or not, by using the prediction model that was learned. For example, the prediction target DB 106 stores therein the worker attendance record data on which the prediction is to be made, and/or the tensor data generated from the worker attendance record data on which the prediction is to be made.

The controlling unit 110 is a processing unit that controls processes performed in the entirety of the learning apparatus 100 and may be, for example, configured by using a processor or the like. The controlling unit 110 includes a tensor generating unit 111, a learning unit 112, and a predicting unit 113. The tensor generating unit 111, the learning unit 112, and the predicting unit 113 are examples of one or more electronic circuits included in the processor or the like; or examples of processes executed by the processor or the like.

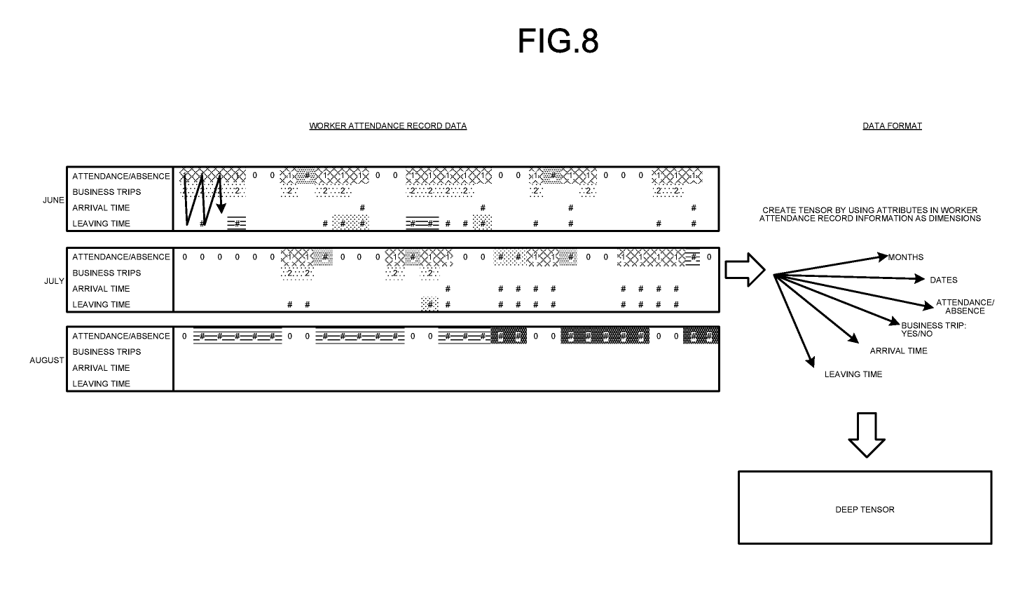

The tensor generating unit 111 is a processing unit that generates the tensor data obtained by creating tensors from the pieces of worker attendance record data. FIG. 8 is a drawing for explaining a tensor data generating process. As illustrated in FIG. 8, the tensor generating unit 111 creates the tensors by using the items “months, dates, attendance/absence; a business trip: Yes/No; arrival times; and leaving times” included in each of the pieces of worker attendance record data as mutually-different dimensions. Further, the tensor generating unit 111 stores the tensor data into the tensor DB 104, so as to be kept in correspondence with either a label (“a medical treatment: Yes” or “a medical treatment: No”) designated by the user or the like or a label (“a medical treatment: Yes” or “a medical treatment: No”) specified from “attendance/absence” in the worker attendance record data. In this situation, the learning process using the deep tensor scheme is performed by using the generated tensor data as an input. In the deep tensor scheme, a target core tensor for identifying a partial pattern of the worker attendance record data that will affect the predicting process is extracted during the learning process. During the predicting process, a prediction is made on the basis of the extracted target core tensor.

More specifically, the tensor generating unit 111 generates the tensors from the worker attendance record data, by using such items that are expected to characterize a tendency of taking an administrative leave of absence (e.g., frequent business trips, long overtime hours, sudden consecutive absences, absences without notice, and a combination of any of these), as the mutually-different dimensions. For example, the tensor generating unit 111 generates a four-dimensional fourth-order tensor by using four elements representing the months, the dates, “attendance/absence”, and “a business trip: Yes/No”. When data from four months is used, the number of elements for the months is “4”. The number of elements for the dates is “31”, because the maximum value for the days in a month is 31. The number of elements for “attendance/absence” is “3” because possible options for attendance and absences are “attended”, “a vacation”, and “a non-business day”. The number of elements for “a business trip: Yes/No” is “2”, because the options are “a business trip: Yes” and “a business trip: No”. Accordingly, the tensor generated from the worker attendance record data is a “4×31×3×2” tensor. The value of each of the elements that correspond to “attendance/absence” and “a business trip: Yes/No” for each of the months and the dates in the worker attendance record data is “1”, whereas the value of each of the elements that do not correspond thereto is “0”. In this situation, it is possible to arbitrarily select the items used as the dimensions of the tensor. It is also possible to determine the items from past examples or the like.

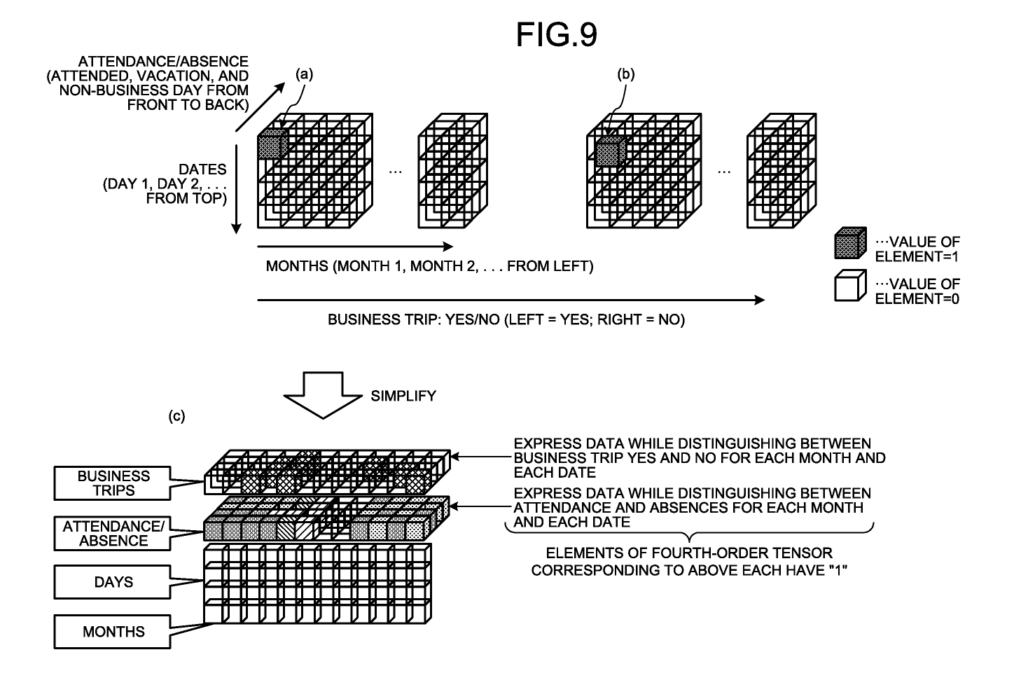

FIG. 9 is a drawing for explaining a specific example of the tensor creating process. As illustrated in FIG. 9, the tensors generated by the tensor generating unit 111 are pieces of data in each of which the months are arranged in the horizontal direction, the dates are arranged in the vertical direction, and “attendance/absence” is arranged in the depth direction, while pieces of data corresponding to “a business trip: Yes” are arranged from the left-hand side, followed by pieces of data corresponding to “a business trip: No”. The dates are sequentially arranged with day 1 at the top. For attendance/absence, the options are arranged in the order of attended, a vacation, and a non-business day, from the front to the back. For example, in FIG. 9, (a) indicates an element where the worker attended on day 1 in month 1 and had a business trip. In FIG. 9, (b) indicates an element where the worker took a vacation on day 2 of month 1 and did not have a business trip.

In the present embodiment, the tensors explained above are simplified and expressed as illustrated in FIG. 9 (c). In other words, the tensor will be expressed in the form of a cube in which the elements of the months, the dates, attendance/absence, and business trip: Yes/No are placed on top of one another. In this situation, business trip: Yes/No will be expressed so as to be distinguished from each other for each of the months and each of the dates, while the categories of attendance/absence will be expressed so as to be distinguished from each other for each of the months and each of the dates.

The learning unit 112 is a processing unit that performs the learning process on the learning model by using the deep tensor scheme, while using the pieces of tensor data and the labels generated from the worker attendance record data as an input. More specifically, similarly to the method explained with reference to FIG. 4, the learning unit 112 extracts a core tensor from the tensor data used as an input (the input tensor), inputs the extracted core tensor to the NN, and calculates an error (a classification error) between a result of the classification process from the NN and the labels appended to the input tensor. Further, by using the classification error, the learning unit 112 learns parameters of the NN and optimizes the target core tensor. After that, when having finished the learning process, the learning unit 112 stores the various types of parameters into the learned result DB 105, as results of the learning process.

The predicting unit 113 is a processing unit that predicts a label of each of the pieces of data subject to the assessment, by using the results of the learning process. More specifically, the predicting unit 113 reads the various types of parameters from the learned result DB 105 and constructs a deep tensor including the neural network or the like in which the various types of parameters are set. Further, the predicting unit 113 reads a piece of worker attendance record data on which a prediction is to be made from the prediction target DB 106, creates a tensor from the read data, and inputs the created tensor into the deep tensor. Subsequently, the predicting unit 113 outputs a result of the predicting process indicating “a medical treatment: Yes” or “a medical treatment: No”. Further, the predicting unit 113 displays the result of the predicting process on a display device or transmits the result of the predicting process to the administrator terminal device.

A Flow in the Process

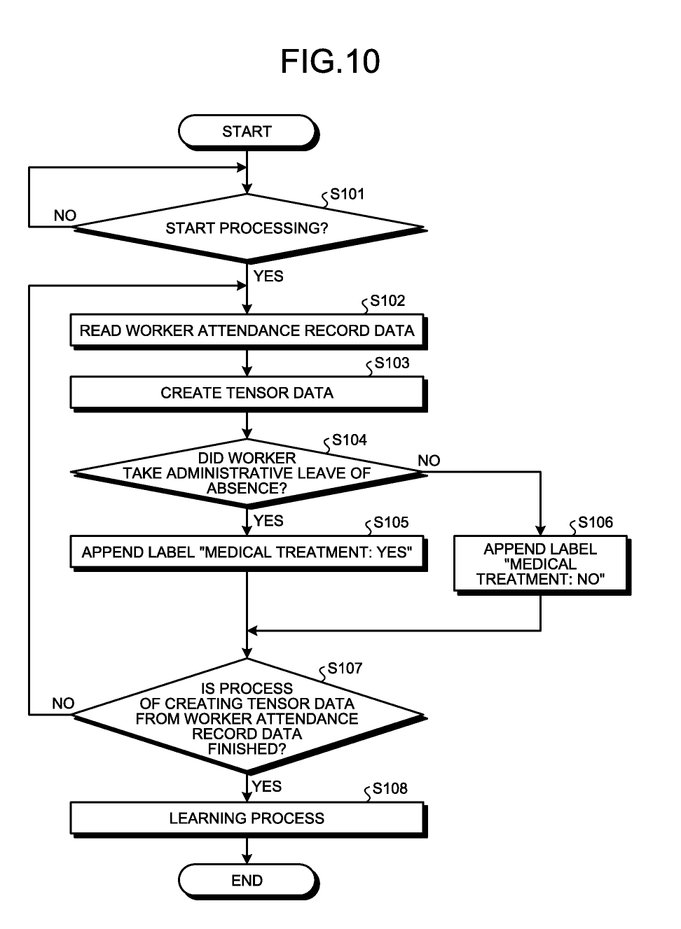

Next, a flow in the learning process will be explained. FIG. 10 is a flowchart illustrating the flow in the learning process. As illustrated in FIG. 10, when it is instructed to start the processing (step S101: Yes), the tensor generating unit 111 reads a piece of worker attendance record data from the worker attendance record data DB 103 (step S102) and creates a tensor data from the read data (step S103).

Subsequently, when the worker corresponding to the piece of worker attendance record data is a worker who took an administrative leave of absence (step S104: Yes), the tensor generating unit 111 appends the label “a medical treatment: Yes” (step S105). On the contrary, when the worker corresponding to the piece of worker attendance record data is a worker who did not take an administrative leave of absence (step S104: No), the tensor generating unit 111 appends the label “a medical treatment: No” (step S106).

After that, when the process of creating tensors from the pieces of worker attendance record data has not been finished, and there is at least one piece of worker attendance record data that has not been processed (step S107: No), the processes at step S102 and thereafter are repeatedly performed. On the contrary, when the process of creating tensors from the pieces of worker attendance record data has been finished (step S107: Yes), the learning unit 112 performs the learning process by using the pieces of tensor data resulting from the tensor creating process (step S108).

Advantageous Effects

As explained above, the learning apparatus 100 is able to perform the machine learning process that takes into consideration the relationships among the plurality of items included in the data subject to the learning process. For example, the learning apparatus 100 is capable of performing the learning process and the predicting process without losing the characteristics of the calendar. It is therefore possible to improve the level of precision of the predicting process.

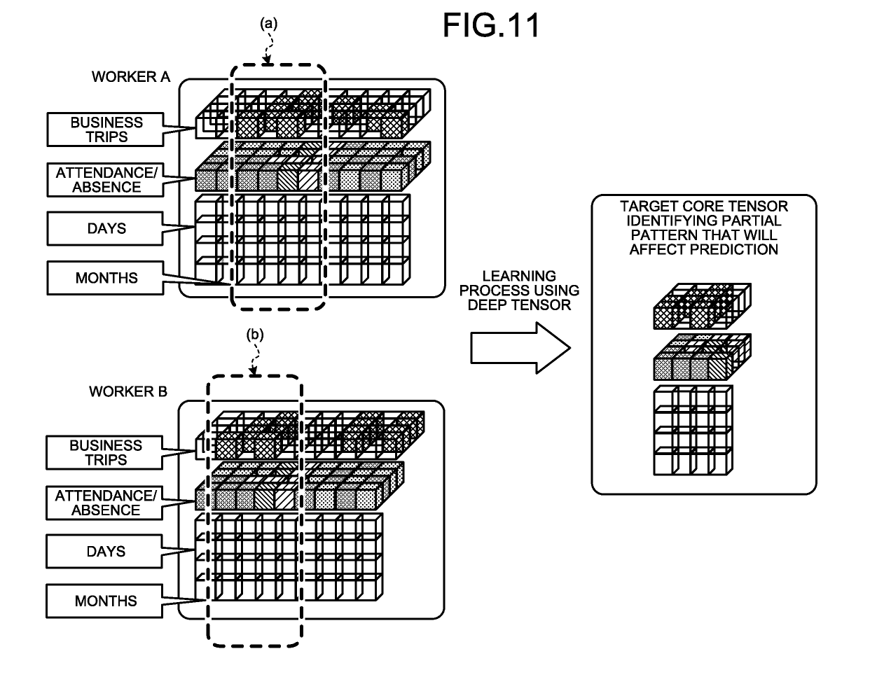

FIG. 11 is a drawing for explaining advantageous effects. As illustrated in FIG. 11, the learning apparatus 100 is able to take into consideration the basic structure of the calendar by incorporating the dimensions corresponding to the months and the dates. Further, by handling the items (attendance/absence and business trips, or the like) in each of the months and the dates in the mutually-different dimensions, the learning apparatus 100 is also able to take into consideration the differences among the items. Furthermore, because each of the elements of the tensor corresponds to a combination of the items in each of the months and the dates, the learning apparatus 100 is able to bring the items on mutually the same date in association with each other.

Accordingly, even when a partial pattern that will affect the predicting process and is extracted as indicated with (a) in FIG. 11 is present in a location different from the location of (a) in FIG. 11, as indicated with (b) in FIG. 11, it is possible to recognize that the partial pattern is the same. It is therefore possible to improve the level of precision in the learning process and the level of precision in the predicting process. In other words, the deep tensor is able to extract the partial structure (the partial pattern of the tensor) of the graph that will contribute to the predicting process as the target core tensor. Accordingly, it is possible to perform the learning process and the predicting process while using the partial pattern of the tensor as an input. As a result, it is possible to recognize the partial pattern (e.g., frequent business trips) in the worker attendance record data, no matter in which month or which date the partial pattern is appearing. It is therefore possible to properly perform the predicting process.

### [b] Second Embodiment

The exemplary embodiments of the present disclosure have thus been explained. However, it is possible to carry out the present disclosure in various different modes other than those in the embodiments described above.

The Learning Process

The learning process described above may be performed as many times as arbitrarily determined. For example, it is acceptable to perform the learning process by using all the pieces of training data. Alternatively, it is also acceptable to perform the learning process only a predetermined number of times. Further, as for the method for calculating the classification error, it is acceptable to use a publicly-known calculation method such as a least squares method. Alternatively, it is also acceptable to use a commonly-used calculation method applied to neural networks. Further, an example of the learning model corresponds to learning a weight or the like of the neural network by inputting the tensors to the neural network, so as to be able to classify events (e.g., “a medical treatment: Yes” and “a medical treatment: No”) by using the learning data.

Further, in the above explanation, the worker attendance record data for the sixth months is used as an example of the data used for the predicting process. However, possible embodiments are not limited to this example. It is acceptable to arbitrarily change the time period to four months or the like. Further, the example is explained in which, with respect to the worker attendance record data for the six months, the label is appended depending on whether or not the worker took an administrative leave of absence within the following three months. However, possible embodiments are not limited to this example. It is acceptable to arbitrarily change the time period to within the following two months, or the like. Further, the tensor data does not necessarily have to be four-dimensional. It is possible to generate tensor data of less than four-dimensional or five or more dimensional. Further, besides the worker attendance record data, it is possible to use data in any format as long as the data is attendance/absence data exhibiting the status of workers arriving at work, leaving work, taking vacations, and the like. Further, it is also possible to use pieces of data starting at mutually-different times such as a piece of worker attendance record data from January to June and another piece of worker attendance record data from February to July.

The Neural Network

In the present embodiment, it is possible to use any of various types of neural networks such as a Recurrent Neural Network (RNN) or a Convolutional Neural Network (CNN). Further, as for the method used in the learning process, it is possible to use any of various publicly-known methods, besides the error backpropagation method. Incidentally, neural networks have a multi-layer structure including, for example, an input layer, an intermediate layer (a hidden layer), and an output layer, while each of the layers has a structure in which a plurality of nodes are connected by edges. Each of the layers has a function called an “activation function”, while each of the edges has a “weight”. The value of each of the nodes is calculated from the values of nodes in the preceding layer, the values of the weights (weight coefficients) of the connecting edges, and the activation function of the layer. As for the calculation method, it is possible to use any of various publicly-known methods.

Further, a learning process in a neural network is a process of correcting parameters (i.e., weights and biases) so that an output layer has a correct value. According to the error backpropagation method, a loss function indicating how much the value of the output layer is different from that in the correct state (a desirable state) is defined with respect to a neural network, so as to update the weights and biases in such a manner that the loss function is minimized, while using a steepest descent method or the like.

A System

Unless noted otherwise, it is acceptable to arbitrarily modify any of the processing procedures, the controlling procedures, specific names, and various information including various types of data and parameters that are presented in the above text and the drawings. Further, the specific examples, the distributions, and the numerical values explained in the embodiments are merely examples and may arbitrarily be modified.

The constituent elements of the apparatuses and the devices illustrated in the drawings are based on functional concepts. Thus, there is no need to physically configure the constituent elements as indicated in the drawings. In other words, the specific modes of distribution and integration of the apparatuses and the devices are not limited to those illustrated in the drawings. It is acceptable to functionally or physically distribute or integrate all or a part of the apparatuses and the devices in any arbitrary units, depending on various loads and the status of use. Further, all or an arbitrary part of the processing functions performed by the apparatuses and the devices may be realized by a CPU and a program analyzed and executed by the CPU or may be realized as hardware using wired logic.

Hardware

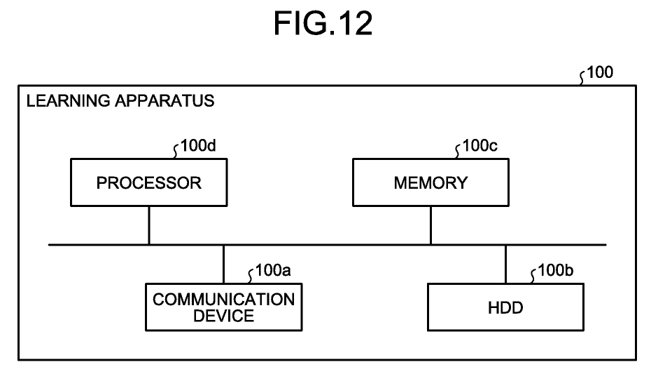

FIG. 12 is a drawing for explaining an example of a hardware configuration. As illustrated in FIG. 12, the learning apparatus 100 includes a communication device 100a, a Hard Disk Drive (HDD) 100b, a memory 100c, and a processor 100d. Further, the functional units illustrated in FIG. 12 are connected to one another via a bus or the like.

The communication device 100a is realized by using a network interface card or the like and communicates with another server. The HDD 100b stores therein a program and databases that bring the functions illustrated in FIG. 5 into operation.

The processor 100d brings into operation the processes that implement the functions explained with reference to FIG. 5 and the like, by reading a program that executes the same processes as those performed by the processing units illustrated in FIG. 5 from the HDD 100b or the like and loading the read program into the memory 100c. In other words, these processes implement the same functions as those of the processing units included in the learning apparatus 100. More specifically, the processor 100d reads the program having the same functions as those of the tensor generating unit 111, the learning unit 112, the predicting unit 113, or the like, from the HDD 100b or the like. Further, the processor 100d executes the processes to implement the same processes as those performed by the tensor generating unit 111, the learning unit 112, the predicting unit 113, or the like.

In this manner, the learning apparatus 100 operates as an information processing apparatus that implements the learning method by reading and executing the program. Further, the learning apparatus 100 is also capable of realizing the same functions as those described in the above embodiments, by reading the program from a recording medium while using a medium reading device and executing the read program. In this situation, the program referred to in the present alternative embodiment does not necessarily have to be executed by the learning apparatus 100. For example, the present disclosure is similarly applicable to situations where the program is executed by another computer or a server or where the program is executed by collaboration of one or more computers and/or one or more servers.

It is possible to distribute the program via a network such as the Internet. Further, the program may be recorded on a computer-readable recording medium such as a hard disk, a flexible disk (FD), a Compact Disk Read-Only Memory (CD-ROM), a Magneto-Optical (MO) disk, a Digital Versatile Disk (DVD), or the like so as to be executed as being read from the recording medium by a computer.

According to one aspect of the embodiments, it is possible to implement the machine learning process while taking into consideration the relationships among the plurality of attributes included in the data subject to the learning process.

All examples and conditional language recited herein are intended for pedagogical purposes of aiding the reader in understanding the invention and the concepts contributed by the inventor to further the art, and are not to be construed as limitations to such specifically recited examples and conditions, nor does the organization of such examples in the specification relate to a showing of the superiority and inferiority of the invention. Although the embodiments of the present invention have been described in detail, it should be understood that the various changes, substitutions, and alterations could be made hereto without departing from the spirit and scope of the invention.

What is claimed is:

1. A non-transitory computer-readable recording medium having stored therein a machine learning program that causes a computer to execute a process, the process comprising:
receiving time-series data including a plurality of items and including a plurality of records corresponding to a calendar;
generating tensor data, based on the time-series data, including a tensor which is set calendar information and each of the plurality of items as mutually-different dimensions; and
with respect to a learning model that performs a tensor decomposition on input tensor data and that inputs a result of the tensor decomposition to a neural network, performing a deep learning process on the neural network and learning a method of the tensor decomposition by using the tensor data as the input tensor data.

2. The non-transitory computer-readable recording medium according to claim 1, the process further comprising:
generating four-dimensional tensor data based on worker attendance record data set with the plurality of items in units of days, while using each of the items corresponding to months, dates, whether or not a worker attended work, and whether or not the worker had a business trip, as the mutually-different dimensions; and
while using the four-dimensional tensor data as the input tensor data, performing the deep learning process on the neural network and learning the method of the tensor decomposition.

3. The non-transitory computer-readable recording medium according to claim 2, the process further comprising:
generating a first piece of four-dimensional tensor data based on a first piece of worker attendance record data including a time period during which an administrative leave of absence was taken;
generating a second piece of four-dimensional tensor data based on a second piece of worker attendance record data including no time period during which an administrative leave of absence was taken; and
performing the deep learning process on the neural network and learning the method of the tensor decomposition so as to be able to classify a first piece of label information and a second piece of label information while using, as supervised data, a first piece of input tensor data using a set made up of the first piece of four-dimensional tensor data and the first piece of label information and a second piece of input tensor data using a set made up of the second piece of four-dimensional tensor data and the second piece of label information.

4. A machine learning method comprising:
receiving time-series data including a plurality of items and including a plurality of records corresponding to a calendar, using a processor;
generating tensor data, based on the time-series data, including a tensor which is set calendar information and each of the plurality of items as mutually-different dimensions, using the processor; and
with respect to a learning model that performs a tensor decomposition on input tensor data and that inputs a result of the tensor decomposition to a neural network, performing a deep learning process on the neural network and learning a method of the tensor decomposition by using the tensor data as the input tensor data, using the processor.

5. A machine learning apparatus comprising:
a memory; and
a processor coupled to the memory and the processor configured to:
receive time-series data including a plurality of items and including a plurality of records corresponding to a calendar;
generate tensor data, based on the time-series data, including a tensor which is set calendar information and each of the plurality of items as mutually-different dimensions; and
with respect to a learning model that performs a tensor decomposition on input tensor data and that inputs a result of the tensor decomposition to a neural network, perform a deep learning process on the neural network and learn a method of the tensor decomposition by using the tensor data as the input tensor data.
